(19) United States
(12) Patent Application Publication
(10) Pub. No.: US 2008/0103363 A1
(43) Pub. Date: May 1, 2008

(54) Device, System, and Method for Programmable In Vivo Imaging

(76) Inventors: Daphna Levy, Carmiel (IL); Dov Avni, Haifa (IL)

Correspondence Address: Pearl Cohen Zedek Latzer, LLP, 1500 Broadway, 12th Floor, New York, NY, 10036, US

(21) Appl. No.: 11/794,539

(22) PCT Filed: Dec. 22, 2005
(86) PCT No.: PCT/IL05/01372

§ 371 Date: Nov. 7, 2007

Related U.S. Application Data
(60) Provisional application No. 60/639,962, filed on Dec. 30, 2004.

(51) Int. Cl. A61B 1/06

(52) U.S. Cl. 600/160

(57) ABSTRACT

Some embodiments of the present invention may relate to devices, systems, and methods for programmable in vivo imaging, for example, a programmable in vivo imager (46), and a method and system for using the programmable in-vivo imager.

### FIELD OF THE INVENTION

The present invention relates to the field of imaging. More specifically, the present invention relates to devices, systems, and methods for programmable in vivo imaging.

### BACKGROUND OF THE INVENTION

Devices, systems and methods for in-vivo sensing of passages or cavities within a body, and for sensing and gathering information (e.g., image information, pH information, temperature information, electrical impedance information, pressure information, etc.), are known in the art.

An in-vivo sensing device may include, for example, an in-vivo imaging device for obtaining images from inside a body cavity or lumen, such as the gastrointestinal (GI) tract. The in-vivo imaging device may include, for example, an imager associated with units such as, for example, an optical system, an illumination source, a controller, a power source, a transmitter, and an antenna. Other types of in-vivo devices exist, such as endoscopes which may not require a transmitter, and in-vivo devices performing functions other than imaging.

In some in-vivo imaging devices, one or more properties or settings of the imager (e.g., image acquisition rate, color settings, illumination settings, etc.) may be pre-programmed, hard-wired or otherwise non-modifiable within the imager of the in-vivo imaging device.

There is a need to allow an efficient modification of a property or a setting of an imager of an in-vivo imaging device, e.g., modification of a property or setting during the operation of the in-vivo imaging device.

### SUMMARY OF THE INVENTION

Various embodiments of the invention provide, for example, a programmable in vivo sensing device, for example, a programmable in vivo imager, and a method and system for using the programmable in-vivo device, as well as a device, system and method for in-vivo imaging. In some embodiments, for example, an imager within an in-vivo imaging device may be programmable, and may include a memory, registers, a table, a mapping table, a lookup table, or other suitable memory unit or storage unit able to store data, parameters, settings and/or properties, e.g., related to the operation of the functionality of the in-vivo imaging device. The memory or other storage device may be, for example, writeable, re-writeable, alterable, modifiable, erasable, etc., after the initial manufacture of the device, and possibly during the operation of the device or when the device is in-vivo.

In some embodiments, a processing unit or controller, typically external to an in-vivo imaging device may set, program or modify a property of an imager or other components of an in-vivo imaging device. In some embodiments, this may allow, for example, setting or modification of properties or parameters of the imager, e.g., during the operation of the in-vivo imaging device and/or upon occurrence of a pre-defined triggering event. An imager may be otherwise controlled, altered or programmed.

Embodiments of the invention may provide an in-vivo imaging device having a programmable imager. The programmable imager may be programmable in substantially real time and/or while the in-vivo imaging device is in-vivo. According to some embodiments, the in-vivo imaging device may include a memory unit to store a parameter used by the programmable imager. According to some embodiments, the memory unit may be within the programmable imager. According to some embodiments, the memory unit may include a register.

According to some embodiments of the invention, the device may include a controller to modify a parameter used by the programmable imager. The controller is typically configured to communicate with the programmable imager. The controller may be configured to write a value into a memory unit which is operatively associated with the programmable imager. According to some embodiments, the controller communicates with the programmable imager through a serial synchronous link.

According to some embodiments of the invention, the programmable imager may be configured to modify its operation based on a parameter stored in the in-vivo imaging device. According to some embodiments, the programmable imager may be programmable by altering a rewriteable memory.

Some embodiments of the invention provide an in-vivo imaging system which may include, for example, an in-vivo imaging device having a modifiable memory unit; and a transceiver, said transceiver being operably connected to a processor. Typically, the imaging device includes an imager. According to some embodiments, the imager may include a modifiable memory unit.

According to some embodiments, the transceiver may be configured to communicate with the in-vivo imaging device through a single-bit bi-directional data line.

According to some embodiments, the in-vivo imaging device includes a transmitter/receiver configured to receive signals from the transceiver. In some embodiments, additionally or alternatively, the imager may be configured to communicate with the transceiver.

According to some embodiments, the system may include a controller to write values into the memory unit and/or to read values from the memory unit. The memory unit is typically configured to store a value of a parameter of the imager. The parameter may be an operational parameter, an illumination parameter, a frame-capture-rate parameter, and/or a fast mode parameter.

According to some embodiments, the invention provides a system including an in-vivo imaging device, the device having an imager operatively connected to a modifiable memory unit, the memory unit able to store a value of a parameter of the imager.

Some embodiments of the invention may provide, for example, a method which includes the step of: modifying in-vivo a parameter used by a programmable imager of an in-vivo imaging device. The parameter may be modified, for example, in response to a triggering event and/or if a pre-defined condition is met. According to one embodiment, the method may include initializing the parameter. According to some embodiments, the method may include writing a value of the parameter into a memory unit within the in-vivo imaging device. In some embodiments, the value may be read after writing. According to some embodiments, the operation of the imager may be modified based on the parameter. In some embodiments, modifying the parameter may be in substantially real time and/or while the in-vivo imaging device is in-vivo.

The method according to some embodiments may include receiving a command to modify the parameter. The command may be received, wirelessly, typically from an external controller.

Embodiments of the invention may allow various other benefits, and may be used in conjunction with various other applications.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a schematic illustration of an in-vivo imaging system in accordance with an embodiment of the invention; and

FIG. 2 is a flow-chart diagram of a method in accordance with an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

It should be noted that although a portion of the discussion may relate to in-vivo imaging devices, systems, and methods, the present invention is not limited in this regard, and embodiments of the present invention may be used in conjunction with various other in-vivo sensing devices, systems, and methods. For example, some embodiments of the invention may be used, for example, in conjunction with in-vivo sensing of pH, in-vivo sensing of temperature, in-vivo sensing of pressure, in-vivo sensing of electrical impedance, in-vivo detection of a substance or a material, in-vivo detection of a medical condition or a pathology, in-vivo acquisition or analysis of data, and/or various other in-vivo sensing devices, systems, and methods.

Furthermore, while a portion of the discussion may relate, for exemplary purposes, to setting, alteration or modification of a property or a parameter of an imager within an in-vivo sensing device, the present invention is not limited in this regard. Embodiments of the present invention may be used in conjunction with, for example, setting, alteration or modification of a property or a parameter of other suitable components of an in-vivo imaging device or in-vivo sensing device, e.g. a sensor, a transmitter, a processor or controller, an illumination unit, or other components. Modification, alteration or setting of properties or parameters of an imager according to embodiments of the invention may be performed not necessarily in the context of in-vivo imaging.

Some embodiments of the present invention are directed to a typically swallowable in-vivo sensing device, e.g., a typically swallowable in-vivo imaging device. Devices according to embodiments of the present invention may be similar to embodiments described in U.S. patent application Ser. No. 09/800,470, entitled “Device And System For In-vivo Imaging”, filed on 8 Mar. 2001, published on Nov. 1, 2001 as United States Patent Application Publication Number 2001/0035902, and/or in U.S. Pat. No. 5,604,531 to Iddan et al., entitled “In Vivo Video Camera System”, each of which is assigned to the common assignee of the present invention and each of which is hereby fully incorporated by reference. Furthermore, a receiving and/or display system which may be suitable for use with embodiments of the present invention may also be similar to embodiments described in U.S. patent application Ser. No. 09/800,470 and/or in U.S. Pat. No. 5,604,531. Devices and systems as described herein may have other configurations and/or other sets of components. For example, the present invention may be practiced using an endoscope, needle, stent, catheter, etc.

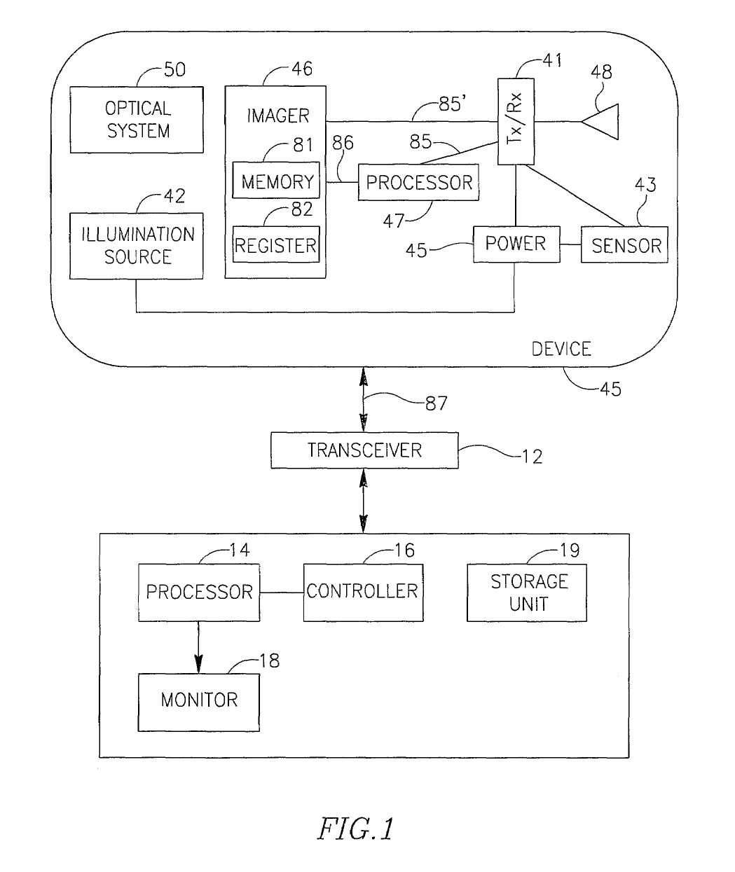

FIG. 1 shows a schematic diagram of an in-vivo imaging system in accordance with an embodiment of the present invention. In one embodiment, the system may include a device 40 having an imager 46, one or more illumination sources 42, a power source 45, and a transmitter/receiver 41. In some embodiments, device 40 may be implemented using a swallowable capsule, but other sorts of devices or suitable implementations may be used. Outside a patient's body may be, for example, an external transceiver 12 (including, for example, an antenna or an antenna array), a storage unit 19, a controller 16, a processor 14, and a monitor 18.

Transmitter/receiver 41 may operate using radio waves; but in some embodiments, such as those where device 40 is or is included within an endoscope, transmitter/receiver 41 may transmit/receive data via, for example, wire, optical fiber and/or other suitable methods. Other known wireless methods of transmission may be used. Transmitter/receiver 41 may include, for example, a transmitter module or sub-unit and a receiver module or sub-unit, or an integrated transceiver or transmitter-receiver.

Device 40 typically may be or may include an autonomous swallowable capsule, but device 40 may have other shapes and need not be swallowable or autonomous. Embodiments of device 40 are typically autonomous, and are typically self-contained. For example, device 40 may be a capsule or other unit where all the components are substantially contained within a container or shell, and where device 40 does not require any wires or cables to, for example, receive power or transmit information.

In some embodiments, device 40 may communicate with an external receiving and display system (e.g., through a monitor on or operatively connected to external transceiver 12) to provide display of data, control, or other functions. For example, power may be provided to device 40 using an internal battery, an internal power source, or a wireless system to receive power. Other embodiments may have other configurations and capabilities. For example, components may be distributed over multiple sites or units, and control information may be received from an external source.

In one embodiment, device 40 may include an in-vivo video camera, for example, imager 46, which may capture and transmit images of, for example, the GI tract while device 40 passes through the GI lumen. Other lumens and/or body cavities may be imaged and/or sensed by device 40. In some embodiments, imager 46 may include, for example, a Charge Coupled Device (CCD) camera or imager, a Complementary Metal Oxide Semiconductor (CMOS) camera or imager, a digital camera, a stills camera, a video camera, or other suitable imagers, cameras, or image acquisition components.

In one embodiment, imager 46 in device 40 may be operationally connected to transmitter 41. Transmitter 41 may transmit images to, for example, external transceiver 12 (e.g., through one or more antennas), which may send the data to processor 14 and/or to storage unit 19. Transmitter/receiver 41 may also include control capability, although control capability may be included in a separate component, e.g., processor 47. Transmitter/receiver 41 may include any suitable transmitter able to transmit image data, other sensed data, and/or other data (e.g., control data) to a receiving device. Transmitter/receiver 41 may also be capable of receiving signals/commands, for example from an external transceiver 12. For example, transmitter/receiver 41 may include an ultra low power Radio Frequency (RF) high bandwidth transmitter, possibly provided in Chip Scale Package (CSP). Transmitter/receiver 41 may transmit/receive via antenna 48. Transmitter/receiver 41 and/or another unit in device 40, e.g., a controller or processor 47, may include control capability, for example, one or more control modules, processing module, circuitry and/or functionality for controlling device 40, for controlling the operational mode or settings of device 40, and/or for performing control operations or processing operations within device 40. According to some embodiments, transmitter/receiver 41 may include a receiver which may receive signals (e.g., from outside the patient's body), for example, through antenna 48 or through a different antenna or receiving element. According to some embodiments, signals or data may be received by a separate receiving device in device 40.

Power source 45 may include one or more batteries. For example, power source 45 may include silver oxide batteries, lithium batteries, other suitable electrochemical cells having a high energy density, or the like. Other suitable power sources may be used. For example, power source 45 may receive power or energy from an external power source (e.g., an electromagnetic field generator), which may be used to transmit power or energy to device 40.

Optionally, in one embodiment, transmitter/receiver 41 may include a processing unit or processor or controller, for example, to process signals and/or data generated by imager 46. In another embodiment, the processing unit may be implemented using a separate component within device 40, e.g., controller or processor 47, or may be implemented as an integral part of imager 46, transmitter/receiver 41, or another component, or may not be needed. The processing unit may include, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In one embodiment, for example, the processing unit or controller may be embedded in or integrated with transmitter/receiver 41, and may be implemented, for example, using an ASIC.

In some embodiments, device 40 may include one or more illumination sources 42, for example one or more Light Emitting Diodes (LEDs), “white LEDs”, or other suitable light sources. Illumination sources 42 may, for example, illuminate a body lumen or cavity being imaged and/or sensed. An optional optical system 50, including, for example, one or more optical elements, such as one or more lenses or composite lens assemblies, one or more suitable optical filters, or any other suitable optical elements, may optionally be included in device 40 and may aid in focusing reflected light onto imager 46 and/or performing other light processing operations.

Data processor 14 may analyze the data received via external transceiver 12 from device 40, and may be in communication with storage unit 19, e.g., transferring frame data to and from storage unit 19. Data processor 14 may also provide the analyzed data to monitor 18, where a user (e.g., a physician) may view or otherwise use the data. In one embodiment, data processor 14 may be configured for real time processing and/or for post processing to be performed and/or viewed at a later time. In the case that control capability (e.g., delay, timing, etc) is external to device 40, a suitable external device (such as, for example, data processor 14 or external transceiver 12) may transmit one or more control signals to device 40.

Monitor 18 may include, for example, one or more screens, monitors, or suitable display units. Monitor 18, for example, may display one or more images or a stream of images captured and/or transmitted by device 40, e.g., images of the GI tract or of other imaged body lumen or cavity. Additionally or alternatively, monitor 18 may display, for example, control data, location or position data (e.g., data describing or indicating the location or the relative location of device 40), orientation data, and various other suitable data. In one embodiment, for example, both an image and its position (e.g., relative to the body lumen being imaged) or location may be presented using monitor 18 and/or may be stored using storage unit 19. Other systems and methods of storing and/or displaying collected image data and/or other data may be used.

Typically, device 40 may transmit image information in discrete portions. Each portion may typically correspond to an image or a frame; other suitable transmission methods may be used. For example, in some embodiments, device 40 may capture and/or acquire an image once every half second, and may transmit the image data to external transceiver 12. Other constant and/or variable capture rates and/or transmission rates may be used.

Typically, the image data recorded and transmitted may include digital color image data; in alternate embodiments, other image formats (e.g., black and white image data) may be used. In one embodiment, each frame of image data may include 256 rows, each row may include 256 pixels, and each pixel may include data for color and brightness according to known methods. For example, a Bayer color filter may be applied. Other suitable data formats may be used, and other suitable numbers or types of rows, columns, arrays, pixels, sub-pixels, boxes, super-pixels and/or colors may be used.

Optionally, device 40 may include one or more sensors 43, instead of or in addition to a sensor such as imager 46. Sensor 43 may, for example, sense, detect, determine and/or measure one or more values of properties or characteristics of the surrounding of device 40. For example, sensor 43 may include a pH sensor, a temperature sensor, an electrical conductivity sensor, a pressure sensor, or any other known suitable in-vivo sensor.

In some embodiments, imager 46 may optionally include a memory unit 81. Memory unit 81 may include, for example, a Random Access Memory (RAM), a Dynamic RAM (DRAM), a Synchronous DRAM (SD-RAM), a Flash memory, a volatile memory, a non-volatile memory, a modifiable memory, a programmable memory, a writeable memory, a cache memory, a buffer, one or more registers, one or more accumulators, one or more lookup tables, one or more tables, one or more maps or mapping tables, a short term memory unit, a long term memory unit, or other suitable memory units or storage units.

In some embodiments, for example, memory unit 81 may be or may include, for example, one or more registers 82, e.g., to store data indicating a current or a desired operational status or parameter of device 40 or a component of device 40. In accordance with some embodiments of the invention, memory unit 81 and/or registers 82 may be, for example, writeable, re-writeable, alterable, modifiable, erasable, etc., after the initial manufacture of device 40, during the operation of device 40, and/or when device 40 is in-vivo. In some embodiments, memory unit 81 and/or registers 82 may include memory which may be altered or written to in ways different from burning a Read Only Memory or setting a hardware switch or jumper, for example, memory which may be written to or altered a plurality of times (e.g., two or more times), memory which may be written to or altered during the operation of device 40 or after initialization of device 40, memory which may be written to or altered using a component of device 40, memory which may be written to or altered using a software operation or component, or the like.

Although memory unit 81 and registers 82 are shown, for exemplary purposes, to be a part of imager 46, the present invention is not limited in this regard. Memory unit 81 and/or registers 82 may be, for example, one or more separate components within device 40, or may be a part of one or more components other than imager 46, e.g., a part of controller or processor 47, or transmitter/receiver 41.

In one embodiment, for example, memory unit 81 may include 16 sixteen-bit registers 82; other number of registers 82 or bits may be used. In some embodiments, memory unit 81 may be internal to imager 46 or integrated with imager 46; in alternate embodiments, memory unit 81 may be external to imager 46, internal to device 40 and operatively connected to imager 46. In some embodiments, for example, memory unit 81 may be integral to processor 47.

In some embodiments, memory unit 81 and/or registers 82 may contain and/or store data, for example, data items, flags, parameters, values, settings, operational parameters or properties (hereinafter, “property data” or “operational parameters”). For example, the property data or operational parameters stored in memory unit 81 and/or registers 82 may indicate, describe or represent a current property of imager 46, a desired property of imager 46, a modification or alteration that needs to be performed to a property of imager 46, a default setting (e.g., a “factory setting”, an original value, or a pre-programmed value) of a property or parameter of imager 46, or other suitable data.

In some embodiments, the property data or operational parameters may indicate, describe or represent, for example, a version identifier, a model identifier, an ID number or an ID string of imager 46, of processor 47, of transmitter/receiver 41, of sensor 43, of device 40, or of other components of device 40. In some embodiments, the property data may include, for example, an indication of one or more pins, switches, units, sub-units or modules which may be enabled, disabled, activated or de-activated in device 40 or in a component of device 40.

In some embodiments, for example, the property data or operational parameters may indicate, describe or represent, for example, a current image capture rate or a desired image capture rate. In one embodiment, for example, a pre-defined bit in a pre-defined register 82 may be set to store the value “1” to indicate an image capture rate of four frames per second or the value of “0” to indicate an image capture rate of two frames per second. Other suitable capture rates may be used, and other bits or registers 82 may be used to indicate a frame capture rate, a data acquisition date, a data transmittal rate, or the like.

In some embodiments, for example, the property data or operational parameters may indicate, describe or represent, for example, a current or a desired operational status of a module or a sub-unit of device 40. For example, one or more pre-defined bits of a pre-defined register 82 may indicate, e.g., using values of “1” and “0”, respectively, whether a module, a component or a sub-unit of device 40 is currently operational or non-operational, whether such module, component or sub-unit needs to be activated or de-activated, or whether a property or operational status of such module, component or sub-unit needs to be set, modified or altered. Such module, component or sub-unit may include, for example, an “Automatic Light Control” (ALC) module which may control illumination source(s) 42, an error correction module, a “sleep” module or mode, a delaying or staggering module or mode, a test mode, algorithms to allow different aspects of the operation of device 40, a module to force a certain pre-defined mode of operation (e.g., to force a fast mode of operation), a module related to external communications, or the like.

In some embodiments, for example, a mapping table having one or more bits may be stored in registers 82 and/or memory unit 81. For example, in one embodiment, one or more bits (e.g., four bits) in register 82 may store values representing a mapping table for exposure time in a normal mode of operation of imager 46, and one or more bits (e.g. two bits) in register 82 may store values representing a mapping table for gain in a normal mode of operation of imager 46. It is noted that in some embodiments, only some of the bits of register 82 may be used to store indications and parameters related to device 40 or its components, and one or more bits of register 82 may remain un-used in certain implementations.

In some embodiments, a first value stored in register 82 or memory unit 81, may be accessed, read or utilized by imager 46 and/or device 40, only if a second stored value corresponds to a pre-defined value.

In accordance with some embodiments of the invention, memory unit 81 and/or registers 82 may store various other modifiable or programmable data or data items.

In some embodiments, a value of a data item stored in memory unit 81 and/or registers 82, e.g., a value of a bit of a register 82, may be set, reset, altered, re-programmed, or modified. The setting or modification may be performed, for example, by imager 46, by transmitter/receiver 41 having control capabilities, by controller or processor 47, by a dedicated controller or unit, or by other component of device 40. In some embodiments, the setting or modification may be performed, for example, based on a triggering event or when a pre-defined condition is met, for example, when a pre-defined time period elapses, or when an external communication signal is received by device 40.

In one embodiment, for example, the values of one or more bits indicating operational parameters and/or operational status of an ALC module, may be set or modified based on an external signal received by device 40. In another embodiment, for example, the values of one or more bits indicating a frame capture rate, a frame transmittal rate, or a fast startup mode, may be set or modified based on a pre-defined condition, e.g., detection of a plurality of consecutive, substantially dark image frames or relatively dark image frames.

In some embodiments, device 40 may receive, or may generate, one or more commands to set, program or modify a value stored in memory unit 81 and/or registers 82. This may allow, for example programming or re-programming of imager 46 or other components of device 40, and/or controlling the operation or the functionality of imager 46 or other components of device 40. In some embodiments, the programming or re-programming may be performed, for example, in substantially real-time, e.g., when device 40 is located inside a patient's body.

In some embodiments, imager 46, transmitter/receiver 41, or processor 47 may include a dedicated module or sub-unit to allow the programming or re-programming of property data or other operational parameters. For example, imager 46 may include a module to allow imager 46 to write data into memory unit 81 and/or register 82, and/or to read data from memory unit 81 and/or register 82. In some embodiments, for example, transmitter/receiver 41 or processor 47 may include a module or a sub-unit to allow writing of data into memory unit 81 and/or register 82, and/or reading of data from memory unit 81 and/or register 82. In one embodiment, for example, memory unit 81 and/or registers 82 may be operatively connected to power source 45, and may receive power from power source 45.

In some embodiments, imager 46 may be operatively connected to transmitter/receiver 41, for example, using a link 85′. Link 85′ may allow serial communication or parallel communication between imager 46 and transmitter/receiver 41. Link 85′ may include, for example, a wired or wireless link.

In some embodiments, transmitter/receiver 41 may be connected through a link 85 to processor 47. Link 85 may include, for example, a wired or wireless link. Link 85 may allow one-way communications or two-way communications (e.g., read-write or bi-polar communications), typically through transmitter/receiver 41, between imager 46 and external transceiver 12. Control information, for example information starting or controlling or ending a process of writing data items to memory unit 81 and/or registers 82, may be transmitted over link 85.

In some embodiments, for example, transmitter/receiver 41 may provide instructions or signals to imager 46 using link 85, or controller or processor 47 may provide instructions or signals to imager 46 using a wired or wireless link 86. For example, signals, typically generated by external transceiver 12, may include an instruction or a signal to modify a property of imager 46, to reset a property of imager 46 to a preset value or a pre-defined value, to modify a resolution of imager 46, to modify an image acquisition rate of imager 46, to modify light settings (e.g., ALC parameters or ALC threshold values) of imager 46, to modify color settings of imager 46, to modify brightness or contrast settings of imager 46, to activate or de-activate a feature or a functionality of imager 46, or to otherwise modify the operation or a property of the operation of imager 46. Link 86 may be or may include, for example, a serial interface which may be used for example, to calibrate imager 46.

In some embodiments, based on, for example, a triggering event, transmitter/receiver 41 or processor 47 may provide to imager 46 the instruction or signal to modify a property of imager 46. The triggering event may include, for example, reception of one or more signals, or determination that one or more pre-defined conditions were met, or the elapse of a pre-defined period of time. The triggering event may include, for example, reception and/or detection by device 40 (e.g., using an optional receiver or transceiver internal to device 40) of a signal transmitted to device 40, instructing device 40 to modify a property of imager 46.

In some embodiments, the triggering event may include, for example, a signal received from sensor 43 indicating measuring or sensing a property which may be above, below, or equal to a pre-defined value. The triggering event may include or may use other suitable functions, calculations, conditions or criteria, which may be alternate or cumulative. In some embodiments, the triggering event may be processed and decided on within device 40, for example by transmitter/receiver 41 or processor 47. According to other embodiments, processing and decisions may be preformed in an external unit, such as, for example, processor 14 and/or controller 16. For example, if transmitter/receiver 41 receives data, such as image data or pH data or temperature data, indicating that device 40 has moved from a first part of a lumen to a second part of the lumen, e.g., from the esophagus into the stomach, then the image capture rate of imager 46 may be changed by modifying a setting stored in memory unit 81 and/or registers 82 of imager 46. In one embodiment, for example, if transmitter/receiver 41 receives data indicating that device 40 has moved from one lumen into another, the gain of imager 46 may be altered. Other setting modifications and programming may be performed.

In some embodiments, imager 46 may perform the desired modification substantially immediately upon reception of the instruction, e.g., or signal from external transceiver 12 through transmitter/receiver 41 or processor 47. Imager 46 may then, for example, delete the data item that indicated the desired modification, or otherwise mark the data item as an instruction which was performed or completed. In alternate embodiments, imager 46 may be required to perform the desired modification at a later time, or repeatedly, or several times, or on a continuous basis. In such cases, the data item may remain in memory unit 81 or registers 82, and imager 46 may access the data item from time to time during its operation to obtain, use, update and/or verify the current value of that data item.

In some embodiments, imager 46 and/or other components within device 40 may be able to access memory unit 81, to read data and/or to write data. For example, transmitter/receiver 41 may write data into memory unit 81, or may read data from memory unit 81. In one embodiment, for example, transmitter/receiver 41 may perform a “read-after-write” operation, such that transmitter/receiver 41 may write a data item into memory unit 81, and then may read the data item from memory unit 81, e.g., to verify that the data item was correctly stored in memory unit 81. Other suitable read and/or write operations may be used or performed, to achieve various functionalities.

In some embodiments, memory unit 81 may be initialized substantially upon the initial activation of device 40. For example, a first set of data may be written into memory unit 81 upon its first use or upon first activation of imager 46. For example, in one embodiment, an initialization process may include copying of values (e.g., “factory settings”) from a Read Only Memory (ROM) or other storage (e.g., located in transmitter/receiver 41, processor 47 or device 40) to memory unit 81. In alternate embodiments, a delete process may be used, a “flush” operation may be performed, a self-test or calibration process may be used, or other initialization operations may be used in relation to the contents of memory unit 81 and/or registers 82.

In some embodiments, link 85 and/or link 86 may be, or may include, a serial synchronous or asynchronous interface, for example, using a clock (e.g., of transmitter/receiver 41 or processor 47) and/or a single-bit bi-directional data line. In one embodiment, registers 82 may be written to and read from through this serial interface.

In some embodiments, a first component of the system may act as a “master” component, and a second component of the system may act as a “slave” component. For example, the “master” component may control the device 40 or components of it, and may provide instructions and/or data to the “slave” component; and the “slave” component may receive instructions and/or data, may execute the instructions, and may send back data to the “master” component. In some embodiments, commands, instructions and/or data may be sent over a typically wireless link 87.

According to another embodiment, imager 46 may act as a “slave”, and processor 47 or transmitter/receiver 41, may act as a “master”, or vice versa. In one embodiment, both a “master” and a “slave” component may control the links 85 and/or 86, but only a “master” component may initiate a command.

In some embodiments, data or commands transferred through links 85, 86 and/or 87 may be in accordance with a pre-defined protocol or format. In one embodiment, for example, the protocol may include the following fields of data: a data item indicating a beginning start of a communication (e.g., a “start bit”); a data item indicating an identifier of the receiving component (e.g., a two-bit “slave” identifier); a data item indicating whether the transferred command is a write command or a read command; a data item indicating whether a read-back operation or a “read after write” operation may be required; an address or other identifier of a register 82; a word data (e.g., a 16-bit word data); and other signals or data. For example, in some embodiments, data transferred through link 87 may include a “charge” field, to charge the link before further operations; a “turnover” field, for tri-stating by the “master” and “slave” components; an “acknowledgement” field (“ACK”) used by a “slave” component acknowledge receipt of a valid command and/or data item; and/or other suitable fields, commands or data items.

In some embodiments, the protocol used for transfer of data over link 87 may include one or more operational modes, for example, three operational modes: a single write operation, a single read operation, and a single write operation with automatic read back.

In some embodiments, an initialization process of memory unit 81 and/or registers 82 may be performed through link 85. For example, the setting initialization may be performed using a pin, a fuse, or a register-only setting. In one embodiment, for example, an initial value set using a pin may be defined as the value written with the pin in its default setting (e.g., a “pullup”); and the initial value of a fuse setting may be defined by the fuse pattern which may be blown, etched or otherwise produced during a production process. It is noted that in one embodiment, a mapping table or data item stored in a register 82, may be entirely accessible using link 85, but may be only partially accessible for initialization using pins. Other means or definitions may be used to initialize one or more data items stored in memory unit 81 and/or registers 82.

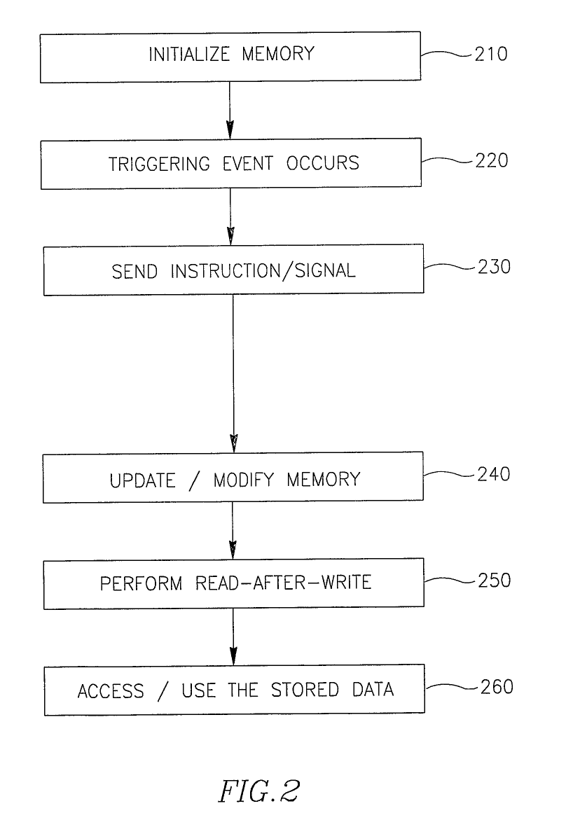

FIG. 2 is a flow-chart diagram of a method of using a programmable imager in accordance with an embodiment of the present invention. The method of FIG. 2, as well as other suitable methods in accordance with embodiments of the invention, may be used, for example, in association with the system of FIG. 1, with one or more in-vivo imaging devices (which may be, but need not be, similar to device 40), and/or with other suitable devices and systems for in-vivo imaging or in-vivo sensing. A method according to embodiments of the invention need not be used in an in-vivo context.

In some embodiments, as indicated at box 210, optionally, a memory unit of an imager of an in-vivo imaging device may be initialized. As a result, initial values may be stored in the memory unit of the imager.

As indicated at box 220, optionally, a triggering event may occur.

As indicated at box 230, optionally, an instruction or signal may be sent, indicating a need to modify or update a property of the imager. For example, the instruction or signal may be sent from external transceiver 12 to imager 46 through link 87, or from processor 47 to imager 46 through link 86.

As indicated at box 240, one or more values stored in memory unit 81 and/or registers 82 of the imager 46 may be programmed, altered, set, reset, updated or modified; for example, a new value may be stored or written in the memory unit, indicating the value of the modified property. It is noted that in some embodiments, optionally, prior data may be erased or over-written with new data. In some embodiments, the operations of box 240 may be performed in-vivo, i.e., when the in-vivo imaging device is in-vivo and/or substantially in real time.

Optionally, as indicated at box 250, a read-after-write operation may be performed, for example, to verify that the update operation of box 240 was performed correctly.

Optionally, as indicated at box 260, during the operation of the imager 46 or the in-vivo imaging device 40, the imager 46 or the device 40 may access data stored in the memory unit 81, may use the stored data, and/or may operate or modify its operation based on to the stored data.

It is noted that some or all of the above-mentioned operations may be performed substantially in real time, e.g., during the operation of the in-vivo imaging device, during the time in which the in-vivo imaging device operates and/or captures images, and/or without interruption to the operation of the in-vivo imaging device.

Other operations or sets of operations may be used. In some embodiments, the method may include operations such as, for example, modifying in-vivo a parameter used by a programmable imager of an in-vivo imaging device; modifying said parameter in response to a triggering event; modifying said parameter when a pre-defined condition is met; initializing said parameter; writing a value of said parameter into a memory unit within said in-vivo imaging device; reading said value after writing said value; modifying the operation of said programmable imager based on said parameter; receiving a command to modify said parameter; modifying said parameter in substantially real time and/or while said in-vivo imaging device is in-vivo; or other suitable operations.

A device, system and method in accordance with some embodiments of the invention may be used, for example, in conjunction with a device which may be inserted into a human body. However, the scope of the present invention is not limited in this regard. For example, some embodiments of the invention may be used in conjunction with a device which may be inserted into a non-human body or an animal body.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

1-35. (canceled)

36. An in-vivo imaging device comprising:
a programmable component wherein said programmable component is configured to modify the operation of said in-vivo imaging device in response to a triggering event.

37. The in-vivo imaging device of claim 36, wherein said triggering event comprises meeting a pre-defined condition.

38. The in-vivo imaging device of claim 36, wherein the programmable component is programmable in substantially real time and/or while the in-vivo imaging device is in-vivo.

39. The in-vivo imaging device of claim 36, wherein the programmable component comprises an imager, an illumination source, a processor or a transceiver.

40. The in-vivo imaging device of claim 36, comprising a memory unit to store a parameter used by the programmable component.

41. The in-vivo imaging device of claim 40, wherein the memory unit is within the programmable component.

42. The in-vivo imaging device of claim 36, comprising a controller to modify a parameter used by the programmable component.

43. The in-vivo imaging device of claim 36, wherein the programmable component is configured to modify its operation based on a parameter stored in the in-vivo imaging device.

44. The in-vivo imaging device of claim 36, wherein the in-vivo imaging device is a swallowable capsule.

45. The system according to claim 36, wherein the imager includes a modifiable memory unit.

46. The system of claim 42, wherein the parameter is an operational parameter and/or an illumination parameter and/or a fast mode parameter and/or a default setting of a parameter of said programmable component.

47. A method comprising:
modifying in-vivo a parameter used by a programmable component of an in-vivo imaging device, wherein said modification of the parameter is in response to a triggering event.

48. The method of claim 47, comprising modifying the operation of said programmable component based on the parameter.

49. The method of claim 47, comprising modifying the parameter in substantially real time and/or while the in-vivo imaging device is in-vivo.

50. The method of claim 47, comprising receiving a command to modify the parameter.

51. The method of claim 47, wherein said triggering event comprises meeting a pre-defined condition.
